(19) United States
(12) Patent Application Publication
(10) Pub. No.: US 2019/0105018 A1
(43) Pub. Date: Apr. 11, 2019

(54) INFORMATION PROCESSING APPARATUS AND INFORMATION PROCESSING METHOD

(71) Applicant: CANON KABUSHIKI KAISHA, Tokyo (JP)

(72) Inventors: Mie Okano, Yokohama-shi (JP); Kouichi Kato, Yokohama-shi (JP); Fumitaro Masaki, Brookline, MA (US)

(21) Appl. No.: 16/150,570

(22) Filed: Oct. 3, 2018

(30) Foreign Application Priority Data
Oct. 10, 2017 (JP) 2017-196845

(51) Int. Cl. A61B 8/08; A61B 5/00

(52) U.S. Cl. CPC A61B 8/5207; A61B 8/085; A61B 8/5215; A61B 5/0095

(57) ABSTRACT

Employed is an information processing apparatus, comprising: an interpretation result acquiring unit configured to acquire a plurality of pieces of interpretation result information resulting from interpretations by a plurality of interpreters of a photoacoustic image originating from photoacoustic waves produced from an object that has been irradiated with light; an interpreter information acquiring unit configured to acquire interpreter information that is information pertaining to the plurality of interpreters; and an information processing unit configured to, based on the interpreter information, acquire information identifying each of the interpreters that performed interpretations corresponding respectively to the plurality of pieces of interpretation result information, and configured to, using this interpreter-identifying information, perform weighting of the plurality of pieces of interpretation result information.

### BACKGROUND OF THE INVENTION

### Field of the Invention

The present invention relates to an information processing apparatus and an information processing method.

### Description of the Related Art

Research is underway into photoimaging, in which characteristic information on an object is acquired by irradiating the object with light. In particular, attention is being directed to the technology referred to as photoacoustic tomography (PAT). Photoacoustic tomography is a technology in which characteristic information on the interior of an object is acquired by detecting the acoustic waves (photoacoustic waves) generated from the object due to the photoacoustic effect when the object is irradiated with light. A photoacoustic tomographic apparatus detects the photoacoustic waves generated within and propagating within an object and, by analyzing the obtained signal, images optical property values in the interior of the object, particularly the absorption coefficient distribution.

In U.S. Patent Application Publication No. 2016/0343132, a method is described in which the location of a tumor within the breast is identified using an ultrasound apparatus and the benign and malignant of the tumor is subsequently assessed by focusing on the blood vessel shape, number, density, and architecture, the amount of hemoglobin, the oxygen saturation, and so forth obtained by a photoacoustic measurement.

As described in U.S. Patent Application Publication No. 2016/0343132, when, for a tumor for which the location has been identified by ultrasound, the benign/malignant of the tumor is to be estimated from the vascular information in the photoacoustic image, the photoacoustic image is first interpreted by an interpreter. When the result of the interpretation is a suspicion of malignancy, a pathology examination is performed and the benign/malignant of the tumor is confirmed.
Patent Literature 1: U.S. Patent Application Publication No. 2016/0343132

### SUMMARY OF THE INVENTION

It is difficult to confirm blood vessel continuity in the interpretation of the vascular image acquired by photoacoustic tomography, particularly in the case of cross-sectional images. In addition, an interpretation technology has not been established since photoacoustic tomography is a relatively new technology. For these reasons, the diagnostic results from the interpretation of photoacoustic tomographic images are readily subject to variability. Thus, one method for improving the diagnostic accuracy is to submit the same photoacoustic image to interpretation by a plurality of interpreters. However, when variability still occurs in the results provided by the plurality of interpreters, it is difficult to rationally assess which interpretation result should be given weight.

The present invention was achieved considering the problem identified above, and an object of the present invention is to provide a technology for carrying out diagnostic support at good accuracy using the interpretation result information when a plurality of interpreters perform an interpretation.

The present invention provides an information processing apparatus, comprising:

an interpretation result acquiring unit configured to acquire a plurality of pieces of interpretation result information resulting from interpretations by a plurality of interpreters of a photoacoustic image originating from photoacoustic waves produced from an object that has been irradiated with light;

an interpreter information acquiring unit configured to acquire interpreter information that is information pertaining to the plurality of interpreters; and

an information processing unit configured to, based on the interpreter information, acquire information identifying each of the interpreters that performed interpretations corresponding respectively to the plurality of pieces of interpretation result information, and configured to, using this interpreter-identifying information, perform weighting of the plurality of pieces of interpretation result information.

The present invention also provides an information processing method comprising the steps of:

acquiring a plurality of pieces of interpretation result information that are results of interpretations by a plurality of interpreters of a photoacoustic image originating from photoacoustic waves produced from an object that has been irradiated with light;

acquiring interpreter information that is information pertaining to the plurality of interpreters; and

performing weighting of the plurality of pieces of interpretation result information based on the interpreter information for the interpreters that performed interpretations corresponding respectively to the plurality of pieces of interpretation result information.

The present invention can thus provide a technology for carrying out diagnostic support at good accuracy using the interpretation result information when a plurality of interpreters perform an interpretation.

Further features of the present invention will become apparent from the following description of exemplary embodiments with reference to the attached drawings.

### BRIEF DESCRIPTION OF THE DRAWINGS

FIG. 1 is a block diagram illustrating the constitution of the apparatus in a first embodiment;

FIG. 2 is a block diagram illustrating the constitution of the server in the first embodiment;

FIG. 3 is a process flow diagram for the first embodiment;

FIGS. 4A to 4C are diagrams each illustrating an example of a display screen;

FIGS. 5A to 5C are diagrams each illustrating another example of the display screen;

FIGS. 6A and 6B are diagrams each showing an input screen for the interpreter in a third embodiment;

FIG. 7 is a diagram illustrating the benign/malignant estimation in the third embodiment; and

FIG. 8 is another diagram illustrating the benign/malignant estimation in the third embodiment.

### DESCRIPTION OF THE EMBODIMENTS

Preferred embodiments of the present invention are described below with reference to the figures. However, the dimensions, materials, shapes, relative positions, and so forth of the constituent components described in the following may be modified as appropriate depending on various conditions and the constitution of the apparatus used with the invention. Thus, the scope of this invention should not be construed as being limited to the description that follows.

The present invention relates to a technology that detects acoustic waves propagated from an object and that produces and acquires characteristic information (object information) about the interior of the object. Thus, the present invention may be embodied as an acoustic apparatus or a method for controlling same, or as an object information acquisition apparatus or a method for controlling same. The present invention may also be embodied as an object information acquisition method or a signal processing method. The present invention may also be embodied as an information processing apparatus or an information processing method, that processes image information and interpreter information for the interior of the object. The present invention may also be embodied as a program that executes these methods on an information processing apparatus provided with hardware resources, e.g., a CPU, memory, and so forth, and/or as a computer-readable non-transitory storage medium that stores this program.

The object information acquisition apparatus according to the present invention contains a photoacoustic apparatus that receives the acoustic waves generated within an object upon the irradiation of the object with light (electromagnetic radiation) and that utilizes the photoacoustic effect to acquire characteristic information about the object as image data. In this case, the characteristic information is characteristic value information for each of a plurality of locations within an object, that is produced using the signal originating with the received photoacoustic waves.

The characteristic information acquired by the photoacoustic apparatus represents the distribution of the generators of the acoustic waves produced by irradiation with light, the initial sound pressure distribution within the object or the absorption coefficient distribution or light energy absorption density distribution derived from the initial sound pressure distribution, or the concentration distribution of the substances constituting a tissue. The substance concentration distribution is, for example, the oxygen saturation distribution, total hemoglobin concentration distribution, or the oxidized/reduced hemoglobin concentration distribution.

The characteristic information, which is object information at a plurality of locations, may be acquired as a two-dimensional or three-dimensional characteristic distribution. The characteristic distribution can be produced as image data that expresses the characteristic information for the interior of the object. The image data is produced, for example, as three-dimensional volume data by image reconstruction.

The acoustic waves referenced by the present invention are typically ultrasound and include the elastic waves referred to as sound waves and acoustic waves. The signal (for example, an electrical signal) converted by, for example, a transducer, from the acoustic waves is also referred to as an acoustic signal or received signal. However, references to ultrasound and acoustic waves in this Specification are not intended as a limitation on the wavelength of these elastic waves. The acoustic waves generated by the photoacoustic effect are referred to as photoacoustic waves and optical ultrasound. The signal (for example, an electrical signal) originating with the photoacoustic waves is also referred to as a photoacoustic signal. The image produced by, for example, image reconstruction, from the photoacoustic signal is referred to as a photoacoustic image.

The initial sound pressure of the photoacoustic waves generated from a region of interest within an object is determined from the Gruneisen coefficient, which is a value that is approximately constant depending on the object, the absorption coefficient of the light absorber in the region of interest, and the amount of light in the region of interest. Accordingly, the absorption coefficient distribution in the interior of an object can be acquired by determining the initial sound pressure distribution using an already known image reconstruction method and using the photoacoustic wave intensity detected with an acoustic wave detector, in combination with determining the distribution of the amount of light based on, for example, the amount of irradiated light. The blood vessel distribution in the interior of an object can be imaged by acquiring the absorption coefficient distribution of hemoglobin, which is present in large amounts in the blood. Moreover, the oxygen saturation distribution in the interior of an object may also be imaged by performing the photoacoustic measurement using light at two wavelengths at which the absorption characteristics of oxyhemoglobin and deoxyhemoglobin differ. It is thought that many new blood vessels are formed around tumor tissue, for example, breast cancer, in order to supply oxygen and nutrients. The performance of tumor-related assessments based on photoacoustic images acquired by photoacoustic measurements is therefore under investigation.

Photoacoustic tomography has a relatively short history among the various modalities used for the diagnostic imaging of the body. Due to this, its interpretation technology has not been firmly established and the accuracy of interpretation and the accuracy of diagnosis depend heavily on the experience and skills of the individual interpreter. Performing diagnoses by reconciling the pieces of interpretation result information provided by a plurality of interpreters has thus been investigated. In addition, for those pieces of interpretation result information for which pathology examination result information (also referred to as pathology diagnosis result information), which is a definitive result from a pathology examination, has been obtained, their use can be contemplated for analysis of the skill level and tendencies of the interpreter, the correction of interpretation results, and the improvement of interpreter skill levels.

### First Embodiment

### Basic Organization

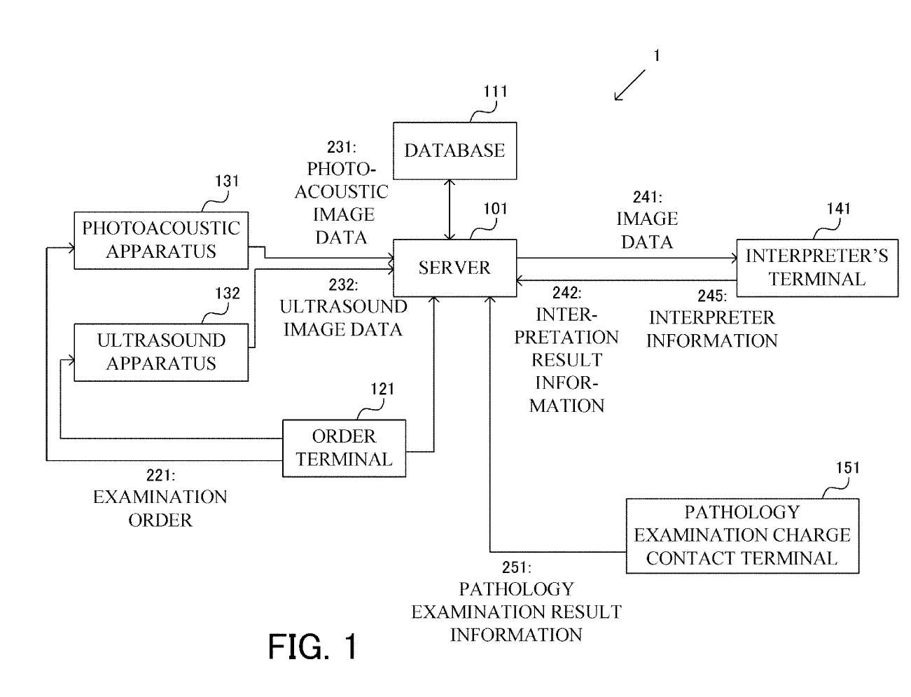

The schematic organization of an exemplary information processing apparatus 1 is given in FIG. 1. The server 101 processes, e.g., the image data provided by the individual modalities, the pieces of interpretation result information, interpreter information, and so forth. It also processes the pathology examination result information when a pathology examination has been performed. The database 111 stores the various data received from the server 101 and outputs same as required.

The order terminal 121 is a terminal where the examiner (for example, a physician) examining the subject (patient) may order examinations by the individual modalities. Photoacoustic tomography is the primary modality in this embodiment. Under operation by an operator (for example, a technician), the photoacoustic apparatus 131 carries out photoacoustic tomographic measurement of an object that is a portion of the subject and transmits photoacoustic image data 231 to the server 101. The figure also shows an ultrasound apparatus that carries out ultrasound echo measurements. Under operation by an operator, the ultrasound apparatus 132 carries out an ultrasound echo measurement on the object and transmits the ultrasound image data 232 to the server 101.

The interpreter's terminal 141 is a terminal where the interpreter (for example, a radiologist or technician) interprets the photoacoustic image of the object. An image is displayed at the interpreter's terminal 141 based on the image data 241 read from the database 111 via the server 101. The findings obtained by the interpreter from the image displayed by a display unit, e.g., a display, are input by the interpreter using an input unit, e.g., a keyboard. This interpretation result report 242 is sent to the server 101 along with the interpreter information 245, which is information that can identify the interpreter. The findings can be exemplified by the presence/absence of a tumor and the location and scale of a tumor, the number of blood vessels and their branching, and so forth. In the organization in this embodiment, a tumor is first identified based on the ultrasound image, followed by interpretation of the vasculature from the photoacoustic image.

A plurality of interpreters are used in the present invention. The system may be provided with a plurality of interpreter's terminals to accommodate the individual interpreters, or a plurality of individuals may use a single interpreter's terminal. Moreover, a single location may be used for the location where interpretation is performed, or remote interpretation may be performed with data sharing.

A pathology examination is ordered from a pathology examination charge contact when the result of the interpretation is the assessment that a pathology examination is required. The pathology examination charge contact inputs the pathology examination result information 251 through the pathology examination charge contact terminal 151 and sends same to the server 101. The pathology examination charge contact is, for example, a physician who has collected tissue, a biopsy technician, or a pathologist who provides findings.

Proceeding as described above, image data captured by the desired modality is stored in the database 111 together with the interpreter information, pieces of interpretation result information, and pathology examination result information.

The organization described in the preceding gives one application example. CT, MRI, an x-ray device, or other may be used as the modality. The examiner, interpreter, and pathology examination charge contact may be different individuals or may be the same individual. The order terminal, interpretation terminal, and pathology examination terminal may also be a single terminal. Conversely, the locations of the order terminal, interpretation terminal, and pathology examination terminal may be dispersed. In addition, the server 101 need not carry out all of the information processing centrally, and all of the data also need not be aggregated in the database 111. The server and the database may be distributed to each functionality or each location.

Specific Organization

The server 101 is typically a DICOM (Digital Imaging and Communications in Medicine) server capable of managing, transmitting, and receiving image data in DICOM format. The server 101 is provided with, for example, a functionality that receives the photoacoustic image data 231 and ultrasound image data 232 and stores same in the database 111, and a functionality that links or integrates the individual image data, the interpreter information 245, the interpretation result information 242, and the pathology examination result information 251. In addition, as described below, it can determine the weighting coefficients for each of the plurality of interpreters and can perform weighting, and/or it can output diagnostic-support information based on the determined weighting coefficients and the interpretation result information.

The following is contemplated for the data stored in the database 111: patient information pertaining to the characteristics of the patient, examination information pertaining to the examination, image data-related image information, interpreter information pertaining to the interpreters, interpretation result information, and pathology examination result information. The data stored in the database 111 is not limited to the DICOM format and may be in a format according to another data standard or may be specific to the apparatus. The following, for example, are stored in the DICOM header in the present embodiment when the DICOM format is used: the interpretation result information, the presence/absence of a pathology examination, the pathology examination result information including a definitive diagnostic result pertaining to the benign/malignant of the tumor, and information pertaining to the skill level of the interpreter, such as the accuracy of putative results for the benign/malignant. The electronic medical record for the patient stores, for example, interpretation result information, information on whether a pathology examination was performed, and the pathology examination result information.

The interpreter information includes information for identifying the interpreter and information pertaining to evaluation of the interpretations of the interpreter. Typically, the former is, for example, the name and/or interpreter ID, while the latter are the tendencies in past interpretations, evaluation values for the skill level, and the results of evaluations by other individuals with regard to skill level.

The interpretation result information is, for example, the presence/absence of a tumor, tumor location and size, opinions with regard to the benign and malignant of the tumor, category classification, whether a pathology examination must be performed, and so forth. More detailed information may also be included in the interpretation result information with regard to, for example, the number and density of blood vessels present in a certain area in the image and the oxygen saturation in this region.

The patient information is, for example, information that identifies the patient, e.g., name and/or patient ID, as well as age, sex, medical history, and so forth.

The examination information is, for example, the modality, date, apparatus information, apparatus operating parameters, and so forth.

The image information is, for example, the image size, bit count, and so forth.

The pathology examination result information is, for example, information that identifies the pathology examination charge contact, e.g., the name and/or ID of the pathology examination charge contact, information on whether a tumor is benign or malignant, and so forth.

The server 101 is advantageously an information processing apparatus (for example, a work station or PC) provided with, e.g., a CPU, memory, communications components, input/output devices, and so forth. This information processing apparatus can be made to function as the server 101 by installing a program that processes DICOM format data. A storage device, e.g., an HDD or SSD, is advantageous for the database 111.

The order terminal 121 is typically an information processing terminal constituting an ordering system. The same information processing apparatus as for the server 101 may be used, or use may be made of a thin client terminal having only a function that presents information to the examiner and a function of receiving input from the examiner.

The photoacoustic apparatus 131 has a light source that generates light; an optical system that guides the light to an object; a probe that receives the photoacoustic waves generated from the object and converts the photoacoustic waves into a photoacoustic signal; and a signal processing unit, which processes and reconstructs the photoacoustic signal and produces photoacoustic image data 231.

A laser, light-emitting diode, flash lamp, and so forth can be used as the light source. The oxygen saturation and substance concentration in the interior of an object can be acquired through the use of a variable-wavelength light source. Optical elements such as optical fiber, mirrors, prisms, lenses, and so forth can be used in the optical system.

The probe is provided with an element, e.g., a piezoelectric element, electrostatic capacitive element, Fabry-Perot element, and so forth, in order to convert the acoustic waves into an electrical signal. The measurement rate and image accuracy are improved by disposing a plurality of elements in the probe in a linear configuration or an array configuration. The probe may be hand held. In addition, a scanning mechanism may be provided whereby the relative position between the probe and the object is changed mechanically.

The signal processing unit contains an AD converter, which converts the electrical signal to digital; an amplifier, which amplifies the electrical signal; and an information processing circuit, which performs a known image reconstruction procedure. The server 101 may carry out the image reconstruction processing rather than the photoacoustic apparatus 131. In this case, the server 101 receives a digital photoacoustic signal from the photoacoustic apparatus 131.

The ultrasound apparatus 132 has a probe, a signal processing unit, and so forth. The probe transmits ultrasound to an object, receives the ultrasound echo reflected from the object, and converts same into an ultrasound signal. The signal processing unit processes and reconstructs the ultrasound signal to produce ultrasound image data 232 that reflects the differences in the acoustic impedance of the tissues in the interior of the object. The same elements as for the photoacoustic apparatus may be used for the probe here. The same probe may be used to transmit and receive the ultrasound, or separate probes may be provided for transmission and reception. The same construction as for the photoacoustic apparatus may also be used for the signal processing unit here. In addition, the same apparatus can carry out the photoacoustic tomography and the ultrasound echo measurement.

The interpreter's terminal 141 is the information processing terminal used by the interpreter. Use may be made of a terminal having a DICOM viewer program installed in the same information processing apparatus as for the server 101, or a thin client terminal, which has only a function that presents information to the interpreter and a function of receiving input from the interpreter, may be used. In the present embodiment, the interpreter manually inputs the interpreter information and interpretation result information through the terminal.

The pathology examination charge contact terminal 151 is the information processing terminal used by the pathology examination charge contact to input the pathology examination result. The same information processing apparatus as for the server 101 or a thin client terminal may also be used for the pathology examination charge contact terminal 151. Moreover, rather than manual entry of the pathology examination result information, a pathology examination apparatus, which has analyzed the sample obtained from the subject by, e.g., injection, may send the pathology examination result directly to the server 101.

The Server and Database

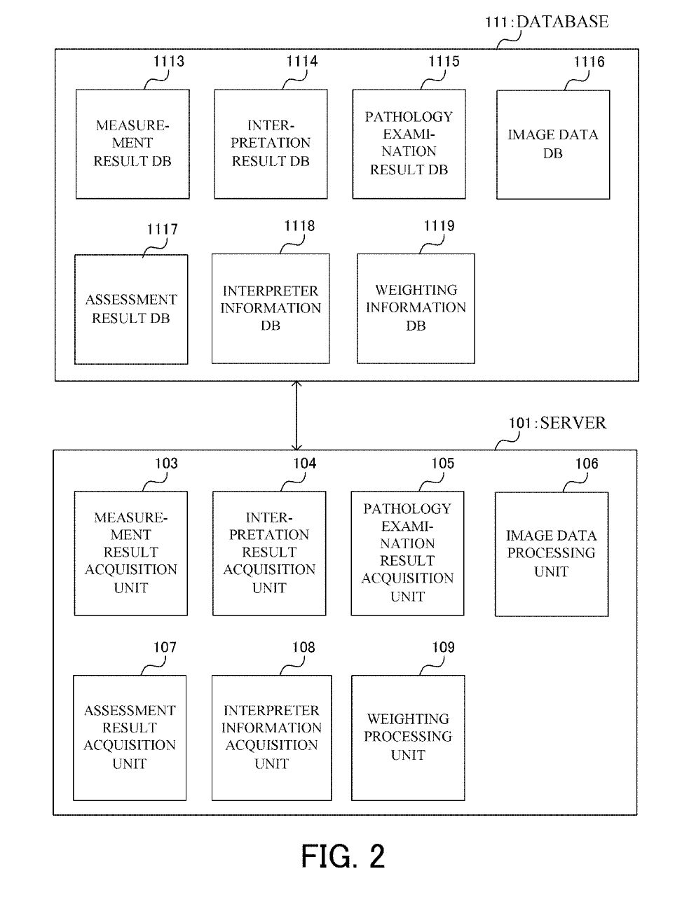

An example of the functional blocks in the server 101 and the database 111 is shown in FIG. 2. This figure is a schematic diagram that gives the functionalities of the server and database as blocks. Accordingly, each block is not necessarily provided with a physical structure, e.g., a circuit, and may be implemented as a program module or a virtual table. A portion of the constituent elements described above (for example, the signal processing unit in the photoacoustic apparatus or ultrasound apparatus, or the server functional component when a thin client is used for the individual terminal) may be incorporated in the server 101. The information processing apparatus of this embodiment may be regarded as including the server, database, terminals, and so forth, or the server component may be regarded as the information processing apparatus.

At the server 101, a measurement result acquisition unit 103 receives the photoacoustic image data 231 and ultrasound image data 232 from the measurement apparatuses. An interpretation result acquisition unit 104 receives the interpretation result information 242 from the interpreter's terminal 141. A pathology examination result acquisition unit 105 receives the pathology examination result information 251 from the pathology examination charge contact terminal 151. An image data processing unit 106 carries out various types of data processing on the image data as necessary (for example, conversion to a prescribed format, correction processing to an image suitable for interpretation). An assessment result acquisition unit 107 acquires the result of the assessment processing pertaining to, e.g., diagnostic support. The assessment result acquisition unit 107 itself may also carry out assessment by functioning as an assessment unit. The interpreter information acquisition unit 108 acquires the interpretation result information and the weighted interpreter information. The weighting processing unit 109 determines the weighting coefficients that are applied to the interpretation result information from the individual interpreters in diagnostic support and performs weighting on the interpretation result information.

At the database 111, a measurement result DB1113 stores the photoacoustic image data and the ultrasound image data. An interpretation result DB1114 stores the interpretation result information. A pathology examination result DB1115 stores the pathology examination result information. An image data DB1116 stores the image data processed by the image data processing unit 106. An assessment result DB1117 stores the result of the assessment processing. An interpreter information DB1118 stores the interpreter information. A weighting information DB1119 stores the weighting coefficients for each interpreter.

The Basic Processing Flow

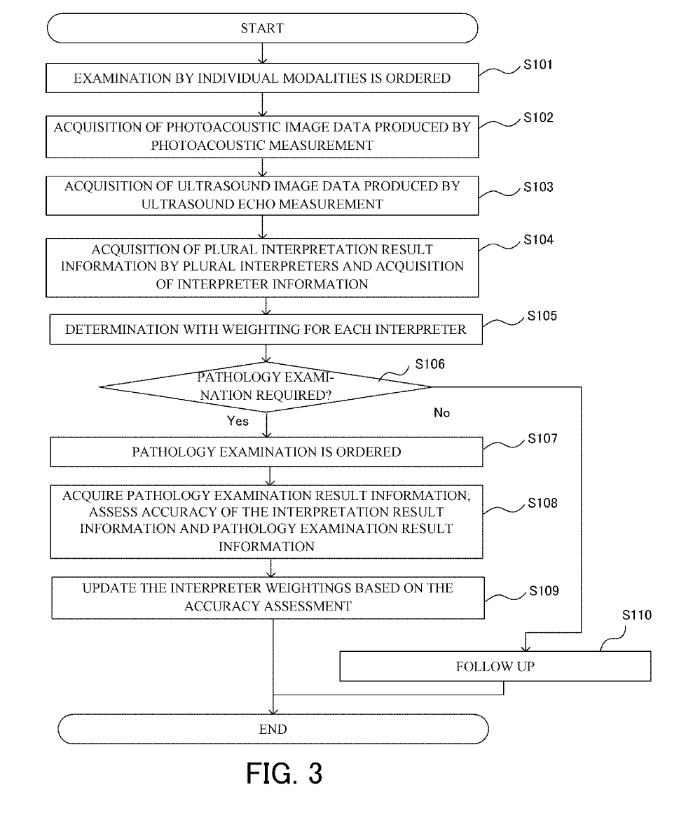

FIG. 3 is a flow chart showing the basic processing flow.

In the step S101, the examiner, having determined that diagnostic imaging on a subject is required, orders an examination with the individual modalities using the order terminal 121 of the ordering system. A technician, having received the order, performs the photoacoustic measurement using the photoacoustic apparatus 131 and produces photoacoustic image data 231. In addition, an ultrasound echo measurement is performed using the ultrasound apparatus 132 to produce ultrasound image data 232.

In the steps S102 and S103, the measurement result acquisition unit 103 of the server 101 acquires the photoacoustic image data 231 and the ultrasound image data 232. The acquired image data is stored in the database 111. Data processing by the image data processing unit 106 may be performed at this time.

Then, image display is performed at the interpreter's terminal 141 based on the image data 241 and interpretation by a plurality of interpreters is required. The photoacoustic image data 231 and ultrasound image data 232 may be used as such as the image data 241, or may be used after, e.g., corrective processing has been carried out. The interpreters perform interpretation and input their findings.

In the present embodiment, the interpreter first refers to the ultrasound image and determines, for the interior of the object, e.g., a breast, the presence/absence of a region where there is the possibility of a tumor and the location and size of this region. Then, referring to the photoacoustic image, the blood vessels are interpreted in the prescribed region and its peripheral region to produce blood vessel information, which is incorporated in the interpretation result information. The blood vessel information is, for example, the number of blood vessels at the periphery of the tumor, the blood vessel density and oxygen saturation in the interior of the tumor, and so forth, and is stored in the database 111 as a portion of the interpretation result information. There are generally numerous blood vessels in the periphery of a malignant tumor. In addition, the blood in the interior of a tumor is hypoxemic. Thus, the benign and malignant of a tumor can be estimated using the number of blood vessels and blood vessel density around the tumor and the oxygen saturation in the interior of the tumor. Other information useful for diagnosis may also be incorporated in the interpretation result information. The Breast Imaging Reporting and Data System (BI-RADS) category estimated from the image may also be incorporated in the interpretation result information.

In the flow under consideration, the interpreter inputs the number of blood vessels as the interpretation result information. As described above, the interpreter may themselves estimate the presence/absence of a tumor and its benign and malignant and incorporate same in the interpretation result information. In addition, the interpreter may perform only an estimation of the blood vessel count, blood vessel density, or oxygen saturation and, based on this data, interpretation support software installed in the server 101 may then estimate the benign/malignant of the tumor. The interpretation support software can be regarded as causing the server to function as an interpretation support unit.

Each interpreter inputs their findings together with identifying information, e.g., an ID and/or name. In the step S104, the interpretation result acquisition unit 104 of the server 101 acquires the plurality of pieces of interpretation result information 242 provided by the plurality of the interpreters. In addition, the interpreter information acquisition unit 108 acquires the interpreter information 245 associated with each interpretation result information.

In the step S105, the weighting processing unit 109 determines the weighting for each of the interpreters. Here, the weightings are acquired from the weighting information DB1119 using the interpreter identification information as the key. The weighting coefficients for each interpreter are stored in advance in the weighting information DB. Typically, the weightings are determined based on the past experience of the interpreter (for example, the correct response rate, interpretation reproducibility, years of experience, number of interpretations, tendencies, and so forth).

When the correct response rate is used as the interpreter weighting, use can be made of the percentage of the number of previous interpretations for the number of interpretation results by the interpreter that agree with the definitive diagnostic results provided by pathology examination. Calculation may be carried out based on, for example, the 50 most recent interpretations for the number of previous interpretations, or calculation may be carried out based on all previous interpretations. The weighted averaging may be performed such that the contribution from the correct response rate is larger for more recent interpretations. In addition, there is no limitation to the correct response rate, and any information that can reflect the interpretation performance of an interpreter can be utilized for weighting.

Using the plurality of pieces of interpretation result information and the weighting of the individual interpreters, the assessment result acquisition unit 107 produces assessment support information for identification of a tumor and malignant tumor. That is, based on the weightings for the individual interpreters, the assessment result acquisition unit produces a weighted interpretation result information by weighting the blood vessel count present in the individual interpretation result information. In addition, the benign and malignant of the tumor is automatically estimated based on whether the weighted interpretation result information (blood vessel information) exceeds a prescribed threshold, and this is provided to the physician. The threshold may be set by the physician or interpreter, but it may also be determined with reference to past results stored in the database. The benign-versus-malignant estimation need not be carried out on the server side, and the interpretation result information and interpreter weightings may be provided and the final assessment may then be queried. The person making the final assessment is preferably the physician who entered the order, but may also be selected from among the interpreters or may be an individual other than an interpreter.

The server 101 provides the physician with the result of the benign-versus-malignant assessment using a display unit (for example, a display). The requirement for a pathology examination is determined by the physician based on the benign-versus-malignant assessment result and also on the interpretation result information themselves and the interpreter information and the weighting coefficients for the individual interpreters, and the result of this determination is input to the server using an input unit (for example, a keyboard). In the step S106, the assessment result acquisition unit 107 of the server 101 acquires the determination result that has been input and determines the subsequent process.

When a pathology examination is required (S106 is “Yes”), the process proceeds to the step S107 and an order is output to the pathology examination charge contact. When, in the determination by a physician of the necessity for a pathology examination, interpretation support software supports the determination of the necessity for a pathology examination, the assessment result acquisition unit 107 receives the interpretation results from the interpretation support software. In addition, it may also be contemplated that the server 101 will determine the necessity for a pathology examination based on the interpretation result information 242 and will execute an order to the pathology examiner. When, on the other hand, a pathology examination is not required (S106 is “No”), follow up is then indicated (step S110).

Having received an order, the pathology examination charge contact performs a pathology examination and acquires a definitive result and inputs this result through the pathology examination charge contact terminal. The pathology examination result acquisition unit 105 of the server 101 acquires the pathology examination result information 251 in the step S107. The benign/malignant tumor information obtained as the definitive diagnosis is incorporated in the pathology examination result information. The pathology examination result information is stored in the pathology examination result DB.

In the step S108, the server 101 compares the acquired pathology examination result information 251 and the interpretation result information 242 and assesses the accuracy of the interpretations. The result of the accuracy assessment is stored associated with the interpretation result information and the interpreter information. Any method may be used to associate the data. The individual data may be stored in a separate table and associated by key, or the data tables may be integrated. When a malignant tumor has been estimated by the interpretation, the image data, interpretation result information, interpreter information, pathology examination result information, and accuracy of the benign/malignant estimation result are stored linked in the database.

In the step S109, the weighting processing unit 109, based on the result of the accuracy assessment, updates the weighting information DB1119 so as to increment the weighting of the interpreters who gave accurate interpretations and decrement the weighting of the interpreters who provided an erroneous interpretation. When this is done, information pertaining to the weighting update may be recorded in the interpreter information DB1118.

Through the flow described above, the weighted interpretation result information, which has been weighted based on the weighting information for the individual interpreters, is produced and assessment by the physician is supported. When the interpretation results from the plurality of interpreters present variability, the use of these weightings makes it possible to consider the interpretation performance of each interpreter and to perform weighting of the interpretation information. It is then possible as a result to rationally execute benign-versus-malignant discrimination on a tumor. As a consequence, the accuracy of diagnostic support using the interpretation result information is improved. In addition to this, the accuracy of the assessment process going forward is further improved when the weightings of the individual interpreters are updated based on an assessment of the accuracy of interpretation.

### Second Embodiment

This embodiment describes a method in which, when the blood vessel count in the plurality of pieces of interpretation result information presents scatter, the benign-versus-malignant estimation of the tumor is performed using a determination of the weightings from the historical correct response rate for each interpreter. As has been described in the preceding, due to the idea that new blood vessels are readily formed in the periphery of tumor tissue, the number of blood vessels in a certain region may be used as interpretation information for estimating the benign and malignant.

In this embodiment, in the step S104 in FIG. 3 three interpreters each independently count the number of blood vessels in the periphery of tumor tissue and input interpreter identification information and the blood vessel count using a single personal computer. The identification information should be information that enables identification of the interpreter, e.g., the name. The interpretation result acquisition unit acquires the blood vessel count. The interpreter information acquisition unit acquires the historical correct response rate using the interpreter ID as the key. Table 1 gives the blood vessel counts contained in the interpretation result information from the three interpreters and the historical correct response rate for each individual interpreter. This correct response rate is the value obtained by comparison of the past benign-versus-malignant assessments by the interpreter with pathology examination results.

TABLE 1

| | interpretation result report | historical correct |
|---|---|---|
| interpreter ID | (blood vessel count) | response rate |
| interpreter 1 | 15 | 0.6 |
| interpreter 2 | 9 | 0.9 |
| interpreter 3 | 27 | 0.2 |

The weighting processing unit calculates the weighted blood vessel count based on the blood vessel counts provided by the individual interpreters and the weights determined from the historical correct response rate. The weighted average blood vessel count is approximately 13 in the present embodiment. Equation (1) is used to calculate the weighted average in this embodiment.

$$\begin{matrix}[ Math . 1 ] & \\ x = \frac{{\sum }_{i = 1}^{n} {w}_{i} {x}_{i}}{{\sum }_{i = 1}^{n} {w}_{i}} & ( 1 )\end{matrix}$$

Here, i is the interpreter ID (1 to 3) and x is the blood vessel count. The weighting is w and in this embodiment is the historical correct response rate.

The weighting processing unit estimates that a tumor is malignant when a threshold, i.e., a predetermined weighted blood vessel count, is exceeded. The prescribed threshold in this embodiment is 16. The estimation result is therefore benign. The result of the benign-versus-malignant estimation result is saved in the database.

According to the method in this embodiment, even when the interpretation results present scatter, the interpretation information can be weighted based on a consideration of the interpretation performance of the individual interpreters. As a result, because useful information can be provided to the diagnostic effort, a beneficial pathology examination is performed in those instances where a pathology examination is required, while an unnecessary pathology examination, which is accompanied by a biopsy, is not performed when a pathology examination is unnecessary.

### Modification Example

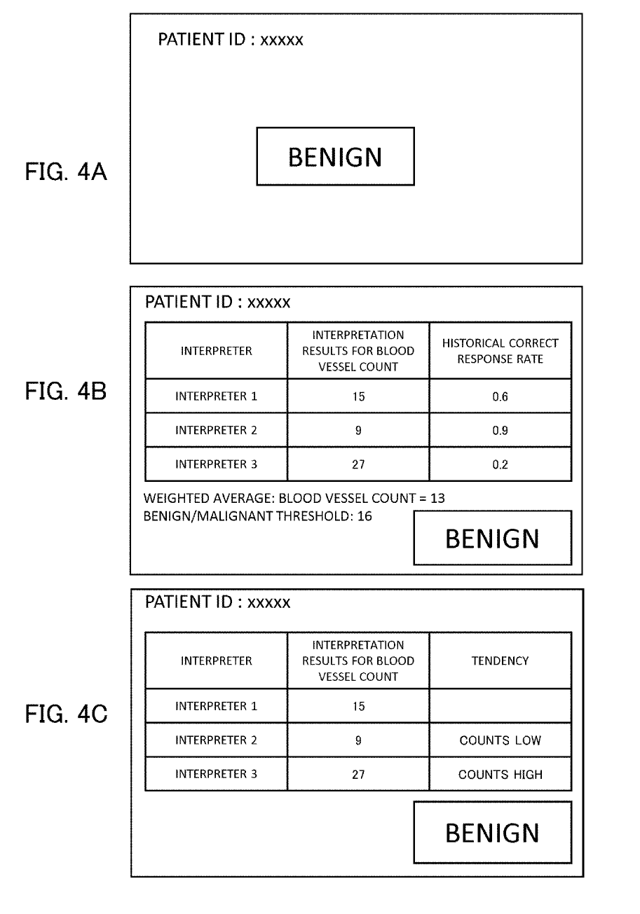

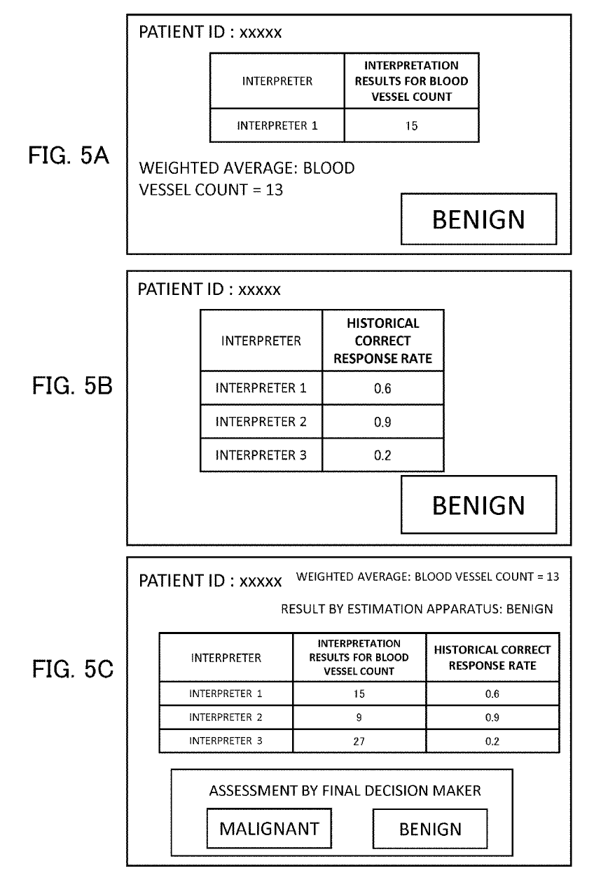

The server preferably also displays it on a display unit, e.g., a display, in a format that enables checking by the physician or interpreter, in addition to storing information, e.g., the benign-versus-malignant assessment result, in the database. FIGS. 4A to 4C and FIGS. 5A to 5C are diagrams for illustrating examples of the display screen.

The patient ID and the benign-versus-malignant estimation result are displayed in FIG. 4A. For example, this screen is displayed on the display unit of the order terminal or interpreter terminal when the physician or interpreter checks the medical record. FIG. 4B is an example in which both the estimation result and the data forming the basis for this estimation are displayed. Thus, the interpretation result information and correct response rate for each individual interpreter, the weighted average value, and the prescribed threshold are also itemized as shown in Table 1.

FIG. 4C is an example of the display of information, stored in the interpreter information DB, that indicates the interpretation tendencies for each interpreter. Individual differences among interpreters readily occur here because the number of blood vessels is counted while checking for blood vessel continuity. For example, when a certain interpreter tends to count too many blood vessels, the estimation result based on the interpretation result information from this interpreter will tend toward a finding of malignancy. In the example in FIG. 4C, the tendencies or idiosyncrasies of the interpreters are therefore displayed in order to reduce the influence of individual differences on the estimation result.

Information indicating the interpretational tendencies can be exemplified by the following: tendencies in the evaluation of the blood vessel count (counts too high, counts too low), reproducibility of interpretation (reproducibility when interpreting the same image is high/low), and tendencies in the final assessment (tends to evaluate as benign/tends to evaluate as malignant).

FIG. 5A gives the interpretation result information for an interpreter 1 and the weighted average for the plurality of interpreters. Such a screen can be used to check the interpretation result information and interpretation performance of a particular interpreter. FIG. 5B gives the correct response rate for each interpreter. A screen conforming to a particular objective can be displayed by limiting the items as shown in FIG. 5A and FIG. 5B. FIG. 5C is an example in which, in addition to the items described above, a message is displayed that queries the final decision maker for a final assessment.

Assessment by the physician can thus be beneficially supported in accordance with this modification example by displaying the interpretation result information by the plurality of interpreters along with the interpreter information and the estimation results.

### Third Embodiment

A method is described in this embodiment in which the weighting processing and benign/malignant assessment are conducted considering the interpretation performance, for the case in which a plurality of interpreters, for whom differences in interpretation performance are assumed to exist, each perform interpretation. The following attributes are assumed for the interpreters: a technician lacking a medical license and a physician interpreter who has a medical license. The target for interpretation in this embodiment is the blood vessel count and the number of blood vessel branches.

In this embodiment, input of the interpreter ID (or name) is required first when the interpreter starts input from the interpreter's terminal. When this is done, the determination is made, based on the interpreter ID, as to whether the interpreter is a technician or a physician interpreter: when the interpreter is a technician, the interpretation result input screen at the display unit at the interpreter's terminal is displayed in a technician mode; when the interpreter is a physician, the interpretation result input screen at the display unit at the interpreter's terminal is displayed in a physician interpreter mode. The assessment of physician participation may be carried out by the server, which has received the interpreter ID from the interpreter's terminal, with reference to the interpreter information DB, or may be carried out by the interpreter's terminal on which interpreter information has been prestored.

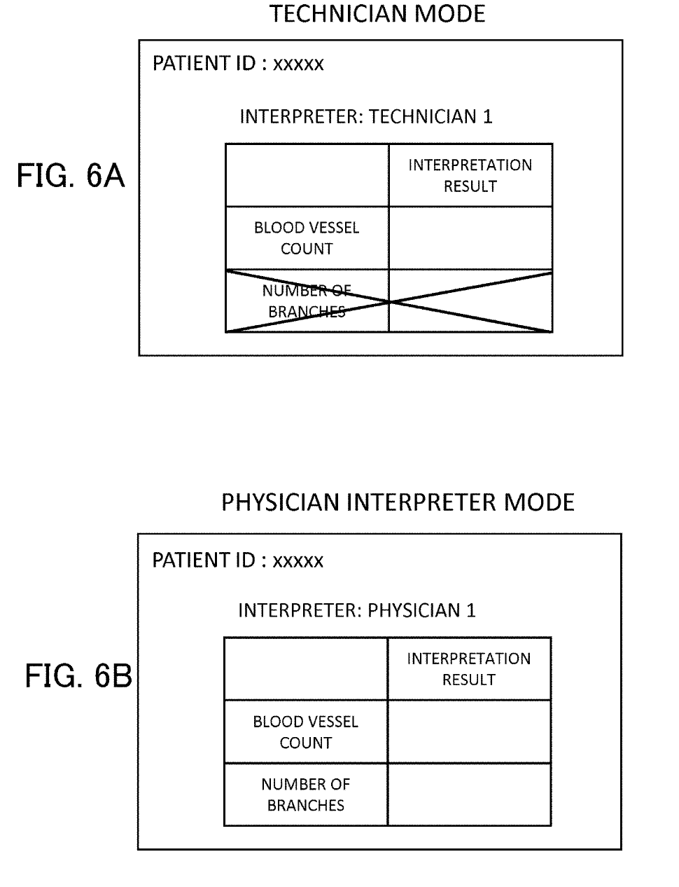

When the interpreter is a physician, there is then a high potential for appreciation of the condition of the blood vessels at a tumor based on experience, e.g., surgical experience. Thus, both the blood vessel count and the number of blood vessel branches can be input in the interpretation result input screen set in physician interpreter mode, as shown in FIG. 6B. When, on the other hand, the interpreter is a technician, just the blood vessel count can be input, as shown in FIG. 6A. Thus, the attributes of the interpreter are acquired from the interpreter information, and the input of the number of blood vessel branches is restricted when the interpreter is not a physician. In the example under consideration, the threshold for the blood vessel count is 16 and the threshold for the number of blood vessel branches is 5, and an estimation of malignancy is made when either of these numerical values is exceeded.

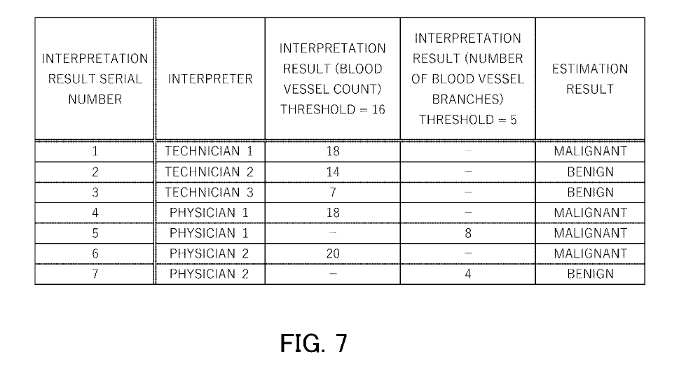

The interpretation results for three technicians and two physician interpreters are given in FIG. 7. The column at the left side gives the serial number for the interpretation results, and the total number of interpretation results, considering both the blood vessel count and the number of blood vessel branches and without consideration of the interpreter, is seven. In this example, the benign/malignant assessment is made based on the majority occurrence in the total number of interpretation results. The second column gives the interpreters (technicians 1 to 3 and physicians 1 and 2). The third and fourth columns provide the interpretation results. The column at the right side gives the individual benign/malignant estimation results, which are obtained by comparing the particular interpretation result with the threshold given above. The final estimation result is malignant since there are three benign estimation results and four malignant estimation results.

The items, e.g., the image data, interpretation result information, information indicating the presence/absence of a pathology examination, pathology examination result information, accuracy of the benign/malignant estimation result, and so forth are linked and saved in the database. The correct response rate in the interpreter information is updated in conformity with the accuracy of the benign/malignant estimation result.

By changing the inputtable information by switching the mode depending on whether the interpreter is a technician or a physician interpreter, this embodiment enables a better weighting based on a consideration of the interpretation performance of the interpreter. The result is that the discrimination of the benign and malignant of a tumor can be carried out rationally.

### Modification Example

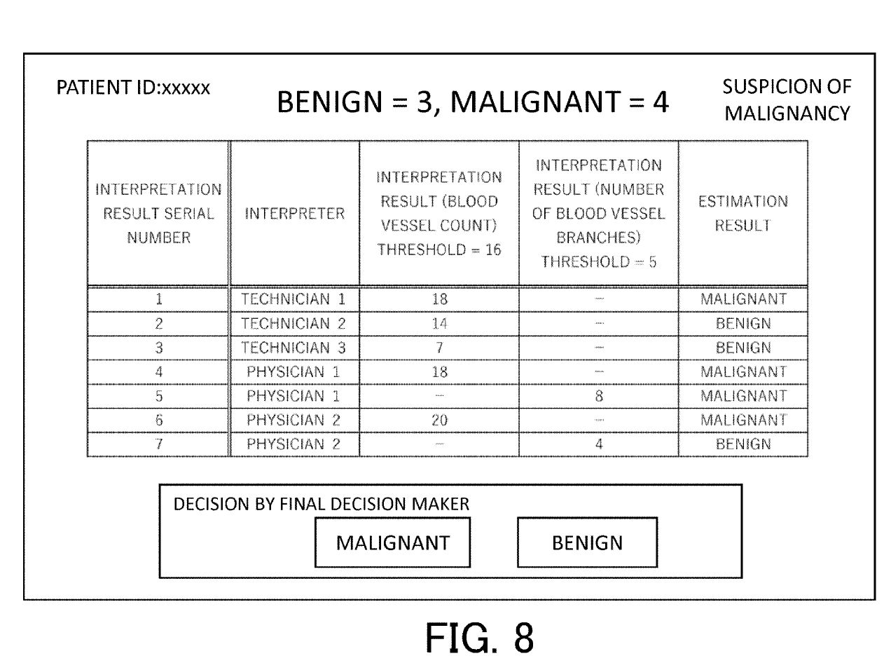

This embodiment was performed on the server side up to and including the final estimation. However, it may be stopped at just the presentation of the information to allow, for example, a physician, to make the final estimation. FIG. 8 is a screen for prompting a final decision and provides the physician with the information shown in FIG. 7 and with only the statement that there is a “suspicion of malignancy” as a result of a majority determination. The final decision maker may be determined in advance or a suitable final decision maker may be selected with reference to, for example, the interpreter information DB. A suitable final decision maker would be, for example, a physician interpreter with many years of experience.

In addition, weighting may be performed in this embodiment based on, for example, the correct response rate for each technician and physician interpreter who performed the interpretation, as in the first embodiment. This enables an even more suitable assessment in conformity with the performance and attributes of the interpreters.

### Fourth Embodiment

This embodiment describes a method, using a plurality of interpreters, in which the benign/malignant estimation is made based on the scatter in the interpretation results when the same image is interpreted. Three interpreters are involved, and the blood vessel count is the target of the interpretation.

The three interpreters perform remote interpretation using respective interpreter's terminals at different locations. Any location may be used for the interpretation as long as the location is communicably connected to the benign/malignant estimation apparatus and may be, for example, a reading facility or the interpreter's home. The interpreter enters their interpreter ID or name and the blood vessel count provided by interpretation, and these are transmitted to the server. When all of the pieces of interpretation result information have been transmitted, the server determines the weightings for the interpretation results with reference to the interpretation reproducibility for each interpreter that is stored in the interpreter information DB, and calculates the weighted blood vessel count and displays same.

Here, “reproducibility” indicates the scatter in the interpretation results that is produced when the same image is interpreted, i.e., the standard deviation (σ). In this embodiment, the weightings are acquired using the standard deviation for the blood vessel count when in the past the same image has been interpreted a plurality of times. This weighting is calculated using equation (2).

$$\begin{matrix}[ Math . 2 ] & \\ {w}_{i} = \frac{1}{{σ}_{i}^{2}} ( i = 1 , 2 , … n ) & ( 2 )\end{matrix}$$

Here, i is the interpreter ID (1 to 3). The weighting is w, and σ is the standard deviation.

The interpretation results for the three interpreters are given in Table 2. The blood vessel count interpretation results for the individual interpreters are, respectively, 15, 9, and 27, and the reproducibilities (σ) are, respectively 4, 2, and 7. In this case, the weightings are then 0.06, 0.25, and 0.02, and the weighted average is approximately 11. Since the threshold for the blood vessel count for benign-versus-malignant discrimination is 16, an estimation of benign is made in this embodiment.

TABLE 2

| | interpretation | | |
|---|---|---|---|
| | result report | reproducibility | |
| interpreter ID | (blood vessel count) | (σ) | weighting (1/σ²) |
| interpreter 1 | 15 | 4 | 0.06 |
| interpreter 2 | 9 | 2 | 0.25 |
| interpreter 3 | 27 | 7 | 0.02 |

Since, as described in the preceding, a weighting could be executed in this embodiment that considered the reproducibility as a measure of interpreter performance, the assessments by interpreters with a smaller diagnostic variability make a larger contribution. As a result, a more accurate estimation can be made and unnecessary examinations can then be reduced and the burden on the patient can be lightened, while necessary examinations are performed and not missed. The weighting procedures in the first embodiment and/or second embodiment may be combined with the weighting in this embodiment.

### OTHER EMBODIMENTS

The present invention may also be realized by the implementation of the following process. Thus, this is a process in which software (program) that performs a functionality in or for an embodiment as described in the preceding may be supplied to the system or apparatus via a network or various storage media and a computer (or CPU, MPU, and so forth) in the system or apparatus reads the program and executes same.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-196845, filed on Oct. 10, 2017, which is hereby incorporated by reference herein in its entirety.

What is claimed is:

1. An information processing apparatus, comprising:
an interpretation result acquiring unit configured to acquire a plurality of pieces of interpretation result information resulting from interpretations by a plurality of interpreters of a photoacoustic image originating from photoacoustic waves produced from an object that has been irradiated with light;
an interpreter information acquiring unit configured to acquire interpreter information that is information pertaining to the plurality of interpreters; and
an information processing unit configured to, based on the interpreter information, acquire information identifying each of the interpreters that performed interpretations corresponding respectively to the plurality of pieces of interpretation result information, and configured to, using this interpreter-identifying information, perform weighting of the plurality of pieces of interpretation result information.

2. The information processing apparatus according to claim 1, wherein the information processing unit is configured to, using weighted interpretation result information provided by weighting the plurality of pieces of interpretation result information, produce information that supports diagnosis of the object.

3. The information processing apparatus according to claim 2,
wherein each of the plurality of pieces of interpretation result information is information pertaining to blood vessels in the object; and
the information processing unit is configured to produce information pertaining to a benign/malignant of a tumor in the object.

4. The information processing apparatus according to claim 1, wherein the information processing unit is configured to perform the weighting based on at least any of rates of correct interpretations by the interpreters, interpretation experiences of the interpreters, the number of cases interpreted by the interpreters, reproducibilities of interpretation by the interpreters, and interpretation tendencies of the interpreters.

5. The information processing apparatus according to claim 1, wherein, when the interpreters input the interpretation result information, the interpreter information acquiring unit changes the inputtable items depending on the interpreter.

6. The information processing apparatus according to claim 5, wherein the interpreter information acquiring unit is configured to restrict the input of the number of blood vessel branches in the object when the interpreter is determined not to be a physician based on the interpreter-identifying information.

7. The information processing apparatus according to claim 1, further comprising:
a pathology examination result acquiring unit configured to, when a pathology examination has been performed based on weighted interpretation result information provided by weighting the plurality of pieces of interpretation result information, acquire pathology examination result information that is a result of the pathology examination, wherein
the information processing unit is configured to, based on a comparison of the pathology examination result information and the interpretation result information, update information contained in the interpreter information and pertaining to an evaluation of the interpretations of the interpreters.

8. An information processing method comprising the steps of:
acquiring a plurality of pieces of interpretation result information that are results of interpretations by a plurality of interpreters of a photoacoustic image originating from photoacoustic waves produced from an object that has been irradiated with light;
acquiring interpreter information that is information pertaining to the plurality of interpreters; and
performing weighting of the plurality of pieces of interpretation result information based on the interpreter information for the interpreters that performed interpretations corresponding respectively to the plurality of pieces of interpretation result information.

9. A non-transitory computer readable storage medium that stores a program for causing a computer to execute the information processing method according to claim 8.
